(19) United States
(12) Patent Application Publication
(10) Pub. No.: US 2005/0159679 A1
(43) Pub. Date: Jul. 21, 2005

(54) Method and apparatus for oculomotor performance testing

(76) Inventors: Gary Lynn Harbin, Salina, KS (US); Warren Redden, Gypsum, KS (US)

Correspondence Address: HOVEY WILLIAMS LLP, 2405 GRAND BLVD., SUITE 400, KANSAS CITY, MO, 64108, US

(21) Appl. No.: 10/761,182

(22) Filed: Jan. 20, 2004

(51) Int. Cl. A61B 3/08; A61B 5/103; A61B 5/117

(52) U.S. Cl. 600/587; 128/905

(57) ABSTRACT

The present invention provides improved oculomotor testing devices and pain tolerance testing devices. Certain oculomotor testing devices test parameters including response time, reaction time, and movement time, as well as precision. The devices are adapted for ambulatory as well as semi-ambulatory and non-ambulatory individuals. Methods of using the devices are provided wherein a visual stimulus is provided and the individual is instructed to perform a movement specific to that visual stimulus. Preferably, the device records the movement done in response to the visual stimulus and, with the aid of a computing device, factors out errors and measures the desired parameter. One embodiment of the present invention permits a user to observe a real-time visual feedback of the force exerted on a load cell. The individual can increase or decrease the amount of force exerted in response to the display of force on a visual feedback exhibitor. Such a device can measure the pain tolerance of an individual by correlating the length of time the individual can maintain a certain exerted force on the load cell with their tolerance for pain.

### BACKGROUND OF THE INVENTION

1. Field of the Invention

The present invention is broadly concerned with devices used in physical testing including oculomotor testing and methods of testing using these devices. More particularly, one embodiment of the present invention is concerned with an oculomotor testing device which can measure a variety of parameters associated with oculomotor response and methods of oculomotor response assessment using this device. Still more particularly, the present invention is concerned with an oculomotor testing device which comprises a plurality of panels having switches which are electrically connected to a computing device. The switches are underneath a pad whereby the switches are activated upon weight being applied to the pad. Even more particularly, the method of the present invention is concerned with measuring a response parameter selected from the group consisting of reaction time, movement time, and combinations thereof and generally comprises the steps of providing a visual stimulus, causing a locomotor response to the stimulus in order to generate data representative of at least one of the parameters, and collecting the data. A second embodiment of the present invention is concerned with a touch screen that is electrically connected to a computing device. Touching the screen when prompted by instructions permits the measurement of a response parameter as described above. Another embodiment of the present invention is concerned with a device for measuring pain tolerance. More particularly, the device can be used to measure muscle strength and the amount of time it takes for a muscle to fatigue. Even more particularly, the pain tolerance testing device is concerned with a test subject visualizing the amount of force exerted on the device and attempting to maintain the exerted force above a certain threshold. The present invention is also concerned with methods of using these latter embodiments.

2. Description of the Prior Art

Oculomotor testing has been performed in the past for a variety of different purposes. For example, oculomotor testing has been performed in order to assess athletic performance, drug or alcohol impairment, balance testing, vestibular disorders, coordination, and disorders of the nervous system. Some oculomotor testing consists of measuring one or more of a variety of time sensitive parameters such as reaction time, movement time and precision. Generally, the oculomotor response consists of sensing a visual stimulus, processing the stimulus, and deciding on a course of action, followed by the movement time which is strongly influenced by coordination or precision of movement.

One device used in the past for oculomotor testing was proposed by Harbin, et al., in Evaluation of Oculomotor Response in Relationship to Sports Performance, 21 Medicine and Science in Sports and Exercise, Vol. 3, pages 258-262 (1989), the teachings and contents of which are hereby incorporated by reference. The device consisted of a response board having five depressable square panels which were connected to pressure activated constant off switches. Each depressable panel was placed above a single switch. A test subject would begin the testing routine by standing on the center square panel whereupon they would be prompted to move to a specific panel and return to the center panel. The device could then measure the total elapsed time between the prompting and completion of the movement. In other words, as soon as the prompt was given, the subject moved to the desired panel and then moved back to the center panel. As soon as the subject's weight was fully returned to the center panel, the time was recorded. This device was deficient in many respects including: 1) There was only one switch per panel which required the test subject to step into the center of the panel in order to insure that the switch would activate and send a signal to the computer collecting the data. If the subject placed all of their weight on the perimeter of the square, the switch might not be activated. Similarly, when returning to the center panel, the switch may not have been activated until the subject's full weight was placed near the center of the center panel. 2) When a series of prompts were given, the device could not record and/or compare differences between the time it took to move in any one specific direction and any other specific direction. 3) If the subject incorrectly moved to the wrong panel, the entire movement had to be repeated before the testing could continue. Additionally, the time taken up by the incorrect movements was merely added in to the total time and was not factored out. Thus, incorrect movements could skew the results. For example, if the prompt instructed the subject to move to a red square panel and the subject moved to a blue square panel, the prompt would be repeated until the subject moved to the red square panel and back to the center panel. Thus if the testing consisted of the total elapsed time required for a series of prompts followed by movements, an error may distort the actual results as the time taken during these movements would be added to the total time.

What is needed in the art is a device which permits more accurate testing and which can measure any one or any combination of testing parameters. What is further needed in the art is a device which can record and factor out errors. What is still further needed is a device that measures each movement and movement direction individually as well as in total. Even further needed is a device which can perform testing for non-ambulatory and semi-ambulatory individuals. Finally, what is needed is a device which is more sensitive to pressure changes and which can be easily disassembled for portability and storage.

With respect to the latter embodiments, what is needed is a safe method of measuring the force exerted during an isometric exercise. What is further needed is a method and apparatus for measuring the force exerted during an isometric exercise and providing real time visual feedback to the test subject.

### SUMMARY OF THE INVENTION

The present invention overcomes the problems outlined above and provides devices useful for testing oculomotor response and pain tolerance in individuals as well as methods for measuring different parameters associated with oculomotor response and pain tolerance.

As used herein, the following definitions apply: “Response time” refers to the total time required from the onset of a stimulus to completion of a task. It can be divided into two separate components: reaction time and movement time. “Reaction time” refers to the interval from onset of the stimulus to the initiation of movement and it is further divided into sensing time and decision time. “Movement time” refers to the interval of time from beginning of movement to completion of the task and it begins when the reaction time ends. It is determined by the ability to accelerate the body or extremity and is strongly influenced by coordination or precision of movement. “Precision” refers to the accuracy of performing a single goal-oriented task with the least number of random moves during the motor performance of that task. “Oculomotor response” refers to the sensing of a visual stimulus, processing the stimulus, and deciding on a course of action (reaction time) followed by the movement time.

One embodiment of the present invention provides an oculomotor testing device generally comprising a testing board which can be of unitary construction or a combination of several board panels placed adjacent one another. When the device is made up of a plurality of board panels, each board panel includes a plurality of switches which can intercommunicate with a computing device. The switches are shiftable from a first position to a second position and these positions generally correspond to an activated or closed position and an inactivated or open position, depending upon whether or not the switch is open or closed. The device also includes a pad which overlies the switches and this pad is shiftable vertically in response to weight being placed on the pad. The switches are designed to close in response to weight being placed upon the pad which compresses the switch from its open position to a closed position. Upon removal of the weight from the pad, the pad and switch return to their original position. Particularly preferred switches utilized in the present invention are ribbon switches such as the CONTROLFLEX ribbon switches manufactured by Tapeswitch Corporation (Farmingdale, N.Y.). Still more preferably, these switches are arranged in a parallel fashion such that activation of any one switch underneath a single pad has the same effect as the activation of any other switch underneath that same pad.

As shown by FIG. 1, a preferred embodiment of the present invention includes four trapezoid-shaped board panels surrounding a single central board panel with the resultant board being in the shape of an octagon. Of course, it is understood that any number or board panels could be used and any desired shape for the resultant device produced by the combination by the board panels is possible. FIG. 1 also shows that the present invention is electrically connected with a computing device such as a personal computer which is in turn connected to a stimulus exhibitor which is in the form of a monitor. However, it is within the invention to have the stimulus exhibitor be any device which can display a visual object. In preferred forms, the computing device will cause the monitor to display a color and this color will be associated with a specific instruction for an individual being tested on the oculomotor testing device of the present invention. In this form of the invention, each of the pads will be a particular color and the test subject will be instructed to step from the central board panel to whichever board panel pad that is the same color as is being displayed on the monitor and then return to the central pad. For example, if the central pad was black and the pad to the right of the test subject in FIG. 1 was yellow, the test subject would be instructed to unweight from the black pad, transfer their weight to the yellow pad, and return their weight to the black pad as soon as the monitor exhibited the visual stimulus of the color yellow. Thus, each of the board panels surrounding the central panel will have a pad which is a different color than the pad associated with the central panel. As any movements are being made, the opening and closing of switches will send data to the computing device which can then be collected and used for a variety of reasons. One preferred arrangement of the pad colors is to have the center square be black, the square to a tested individual's right be yellow, the square to their left be red, the square behind them be green and the square in front of them be blue (all relative to when an individual is standing on the board and facing the visual stimulus exhibitor). It is also noted that the stimulus exhibitor cannot be viewed at the same time as the board panels by the individual using the equipment. This will cause an individual to either remember where the pad is that they are supposed to move to without first looking down or slow their recorded time parameters down by looking at the pads after viewing the stimulus displayed on the monitor.

The board panels of the present invention are shown in greater detail in FIG. 2 which illustrate the sandwich-type construction of the individual board panels. Preferably, each individual board panel includes a perimeter frame enclosing the switches therein. In this form of the invention, the central board panel is in the shape of a square which encloses three strip switches which are spaced approximately equidistant from one another with one switch being centrally located within the square and the other two switches at opposite sides of the square adjacent the frame. An opening is provided such that the wires connected to the switches can exit from the frame and eventually lead to the computing device. The preferred board panels also include a top sheet and a bottom sheet which are placed on top of and below, respectively, the frame with the bottom sheet being adjacent the floor when the device is in position to be used. The top sheet further includes a cut-out portion through which at least a portion of the pad is sized to fit within. This cut-out portion permits relative movement between the top sheet and the pad as the two pieces are not coupled together. The top sheet and bottom sheet can be made of any suitable material with aluminum being particularly preferred. The top sheet and bottom sheet are fastened to the frame using any conventional fastening device or method. A preferred fastener will be screws which extend through the respective sheet and into screw holes provided in each respective board panel frame.

The four trapezoid-shaped board panels also include an interior frame member which is positioned such that the switches are enclosed within the space created by having the interior frame member span between opposite sides of the board panel frame. In the embodiment shown in the figures, the space created is a square similar to that presented by the central board panel.

The wiring of the switches and their connections which ultimately lead to the computing device are schematically illustrated in FIG. 3. As shown in that figure, each of the switches for each individual pad are connected in parallel with one another. The electrical leads from these switches exit each respective board panel through a connector which relays the signals provided by the switches for each board panel to an interface which couples the computing device to the switches. It is preferred that each board panel have a single connector which links the electrical leads from any set of switches in an adjacent board panel and also maintains and distinguishes between signals coming from switches under difference pads. For example, the diagram in FIG. 3 illustrates that three of the strip switches must pass through two other board panels before reaching the interface.

The cross-sectional view illustrated in FIG. 4 provides a more detailed perspective of certain preferred aspects of present invention. The pad preferably includes a base portion which rests upon the switch which in turn is placed upon a switch holder or block. The base portion of the pad is located beneath the top sheet or cover for the board panel. The pad further includes a raised portion positioned atop the base and this raised portion preferably is positioned such that the top surface is on a slightly higher plane than the top sheet of the board panel. In even more preferred forms, the raised portion of the pad includes some type of a traction-increasing substance or surface such that a test subject moving quickly from pad to pad will not slip. Additionally, the preferred construction permits relative movement between the pad and the frame or top sheet such that when weight is placed upon the pad, the pad depresses the switch upon which it sits until the base portion of the pad contacts a shoulder which is cut into the frame. When the weight is removed from the pad, the switch and pad elevate from the shoulder and return to the original position of an unweighted pad.

The board panels are preferably removably coupled with one another such that the board panels do not move relative to one another once the device is assembled for use. One preferred connecting method utilizes cooperative dovetail projections and dovetail recesses on adjacent board panels. These dovetail projections and recesses permit the device to be assembled by inserting a dovetail projection into a cooperative dovetail recess and sliding the board along the track created by the recess. In preferred forms, each of the trapezoid-shaped board panels includes either a dovetail projection or a dovetail recess along each frame member which lies adjacent to another board panel including the centrally located board panel. However, in the alternative embodiment shown in FIG. 2, one of the trapezoid-shaped board panels does not include a dovetail projection or recess along one of the sides which is adjacent to another board panel. The portion of the adjacent board panel which lies adjacent to the frame portion of the board panel without the dovetail connector is also devoid of any dovetail projections or recesses. In order to secure these two board panels together, a pin-shaped projection is provided on one of the panels and inserts into a hole on the adjoining panel and this pin is ultimately secured into place via a locking screw which is perpendicular to the pin and is inserted into the adjoining frame member and turned to lock the pin in place.

The device of the present invention is useful in many respects including testing of a time-sensitive parameter such as reaction time, movement time, precision or coordination. Such testing can detect biomechanical imbalances, evaluate rehabilitation progress of injuries and the like, evaluate progression of neurological disorders such as Alzheimer's and multiple sclerosis, test for vestibular disorders, determine drug or alcohol impairment, determine the effect of medication and medication changes including dosage changes, testing child locomotor skills (pre-kindergarten-12th grade), geriatric evaluation, and predicting athletic and workplace potential in any individual.

A method of using this embodiment of the invention generally comprises the step of providing a visual stimulus wherein the visual stimulus causes a locomotor response in the individual being tested and this response generates data representative of a time-sensitive parameter such as reaction time, movement time and combinations thereof. Preferably, the invention also includes the step of collecting the data in order to measure the desired parameter. These steps can be repeated for a number of movements, preferably at least four movements, still more preferably between four and 10,000 movements, still more preferably at least eight movements, even more preferably between eight and 100 movements, and most preferably between about eight and 40 movements. The data generated by the opening and closing of the switches in response to weight being placed on any of the respective pads is collected by the computer and this data can be processed for any of the desired parameters. Furthermore, this data can be compared with other similar data collected from the same individual, a different individual, a known standard, a specific population of individuals, or the population as a whole.

Testing can further include a second testing trial similar to the first which is preformed after the first testing period. Preferably, there is a rest period between the first testing period and the second testing period. Such a rest period can be for any length of time, preferably at least one second, more preferably at least one minute, still more preferably between 1-5 minutes, and most preferably between about 2-7 minutes. This pattern can be repeated for as many testing trials as desired and it is preferred that a rest period be included between each testing period.

In a second embodiment of the present invention, an elongate board having two pads thereon is provided, together with a visual stimulus exhibitor and a computing device. As with the embodiment described above, the visual stimulus exhibitor and the board are located in different planes of sight so that both cannot be viewed simultaneously by the individual using the embodiment. The construction of the board is similar to the board described above with one exception being that it is smaller and has just two pads. Each pad is preferably colored (even more preferably with the pad to the left being red and the pad to the right being black, relative to an individual standing on the board and facing the exhibitor) and overlies a plurality of switches which are adapted to intercommunicate with a computing device. This intercommunication can be via wireless technology or may involve hard wiring between the computing device and the apparatus. As with the switches described above, the switches are shiftable from a first position to a second position. Preferably, each pad is square and is vertically shiftable in response to weight being placed on the pad. When weight is applied to the pad, the pad shifts downward and causes the switches to become compressed and thereby close. Once weight is removed from the pad, the pad shifts upward and away from the switches and thereby permits the decompression of the switches, leading to their opening. It is preferred that the switches for this embodiment be placed in parallel, as described above. CONTROFLEX ribbon switches are the preferred switches for this embodiment. A preferred video stimulus exhibitor is a conventional computer monitor electrically connected to the computing device. The stimulus displayed by the exhibitor can be anything which provides a distinguishable cue (e.g. colors, sounds, arrows, words, etc) to which a tested individual can respond.

In use, this two-pad embodiment is especially preferred for measuring reaction time and is especially useful for measuring the effect of and monitoring minor brain traumas and the progression of diseases such as dementia, Alzheimer's, Parkinson's, and cerebral palsy. A preferred method of using this embodiment generally comprises the steps of positioning a test subject on the board with one foot on each pad. A countdown is displayed on the exhibitor notifying the tested individual of when the testing period will start. Once the countdown reaches one, the visual stimulus is randomly exhibited by the visual stimulus exhibitor to induce a locomotor response in the individual which activates the switches underlying the pads, thereby generating data representative of a time-sensitive parameter such as reaction time. In preferred forms, the video stimulus exhibitor is a video monitor and the computing device sends a signal to the monitor to provide a specific visual stimulus. Additionally, the monitor screen will preferably turn white after the countdown reaches one and before the onset of the first visual stimulus display. Preferably, the invention also includes the step of collecting the data in order to measure the desired parameter. A preferred locomotor response is lifting a foot off of a specific pad in response to the visual stimulus. These steps can be repeated for a number of times and data collected, as described above. Subsequent trials can also be performed, as described above.

In another embodiment of the present invention, the same measurements can be performed by a non-ambulatory individual. This embodiment generally consists of a stimulus exhibitor, preferably a computer video monitor, and a touch screen monitor that are adapted to intercommunicate with a computing device. The intercommunication can be via wireless technology or may involve an electrical connection (e.g. hard wiring) between the components. The stimulus exhibitor is used to exhibit or display a visual stimulus which prompts an individual using the embodiment to execute a desired locomotor response. In preferred forms, the computing device sends a signal to a video monitor to provide the visual stimulus and this stimulus prompts the tested individual to touch a certain portion of the screen of the touch screen monitor. “Touch screen” refers to devices that communicate with computing devices based on signals generated by touching the screen of a specialized monitor. By touching the screen, the screen sends a signal to the computing device which then computes the desired measurement parameter. The stimulus can be any distinguishable display such as a color, arrow, word, or even a sound. The tested individual responds to this stimulus by touching a portion of the touch screen. The touch screen will always have at least one correct area to touch and may also have at least one incorrect area to touch. In one example of a test using this embodiment, an individual is seated in front of the touch screen monitor and the visual stimulus exhibitor. The touch screen monitor and exhibitor are preferably located in different planes of sight so that the visual stimulus exhibitor and the touch screen cannot be viewed simultaneously by the individual without changing their direction of viewing (e.g. looking up at one and down at the other). The interconnected computing device then sends a signal to the visual stimulus exhibitor to display a selected stimulus and records the time that the stimulus was exhibited. The individual views the stimulus and responds as fast as they can by touching the appropriate area of the touch screen and the computing device records the length of time between the display of the stimulus and the completion of the action.

In a particularly preferred form of the touch screen embodiment, the computing device sends a signal to a video monitor to display a specific color. The touch screen will have an area with the same color thereon and the individual must touch the area to complete the action. The touch screen may also have at least one other area that displays a different color than is shown on the monitor, thereby forcing the individual to select the correct one based on the color. When the individual touches the touch screen, a signal is sent to the computing device which then determines if the correct area was touched. If it was touched, that test repetition does not have to be repeated. However, if an incorrect area was touched, the computing device records the error and repeats the repetition. For repetitions that are correctly completed, the computing device records the time between the onset of the stimulus and the touching of the screen. Of course, this test can be repeated any number of times for any number of repetitions. During testing periods that consist of multiple repetitions, it is preferred for the times between repetitions to be of random lengths so as to prevent anticipating moves by the individual. It is also possible to have the individual perform a series of movements as quickly as possible using this device. For example, the stimulus could exhibit two colors and the individual would have to touch each of the areas on the screen corresponding to the displayed colors as quickly as possible.

In another embodiment of the present invention, a pain tolerance testing device is provided. The device generally consists of a computing device, a visual feedback exhibitor, preferably a conventional computer monitor, and a testing apparatus. The computing device is generally a personal computer which is adapted for intercommunication with the visual stimulus exhibitor and the testing apparatus. The testing apparatus generally comprises a housing and a force-receiving member extending from. The base is preferably weighted or adapted to accommodate weight which will help to hold the device in place during use. Alternatively, the device may be equipped with some attachment or securing devices which would be used to secure this device to another object including a floor, wall, or piece of furniture or equipment. In a particularly preferred form, the device is adapted for connection to one of the board type embodiments described herein via a series of screws, velcro, or the like. The force receiving member is connected with a load cell and is preferably a tube or lever. In preferred forms, the force-receiving member is a tube that includes a telescoping portion that can telescope to different lengths in order to accommodate the testing of individuals of different heights. Once a desired height of the telescoping portion is found, the telescoping portion is locked into place using any conventional locking type device (for example, hand collets, screws, bolts, fasteners, snap-out buttons, etc.) in order to prevent further telescoping and so that the force-receiving member acts as one integral unit that extends into the base. Alternatively, the force receiving member can be of a fixed height or have a plurality of different length tubes or levers that can be attached to the device before use. The load cell is also located within the base and is electrically connected to the force-receiving member and at least one, preferably two, ADAM modules. The first ADAM module receives information from the load cell and transmits the information to the second ADAM module while the second ADAM module receives this information and transmits it to the computing device. In one preferred embodiment, the tube has a handle at one end for a tested individual to grasp and the other end is connected to an anchor or anti-rotate pin which is designed to prevent any relative movement of the tube. Such an anchor also serves to protect the load cell, ADAM modules, and other electronics in the base from being displaced during movement of the force-receiving member. Thus, it is desired for the force receiving member to be stationary within the base and to avoid relative movement between the force-receiving member and the base. The load cell is designed to measure the amount of force exerted on the force-receiving member without any movement of the force-receiving member. The load cell sends data corresponding to the force exerted on the force-receiving member to the first ADAM module. This data is then sent to the second ADAM module which transmits it to the computing device for processing. This data is then transmitted to the visual feedback exhibitor in order to provide a real-time display of the forces exerted on the force-receiving member. In other words, an individual using the device can observe the visual feedback exhibitor display a visual readout of the force exerted on the force-receiving member and immediately observe changes in the amount of force exerted.

In use, the pain tolerance testing device can be used to test an individual's strength, endurance, pain tolerance, progress in physical therapy, progress in the rehabilitation of an injury, recovery from a neurological disorder, as well as determine the effects of a medication, or the extent of a disease or neurological condition in an individual. In preferred forms, an individual will face the visual stimulus exhibitor and grasp the end of the lever or tube with the palm side of their hand being down. The end of the lever or tube will preferably include a handle or grip for the individual to grasp. If an adjustable height device is used, the individual will extend the tube to a point so that it is in a position to hold the individual's arm at a 90° angle relative to their body. The tube is then locked into place in order to prevent relative movement between the tube and the base. The individual is then instructed to pull upward on the tube or lever using as much force as possible. This is designed to measure the individual's maximum strength. Preferably, the individual is able to watch the level of force exerted on the tube or lever on the visual feedback exhibitor. The feedback may be displayed as a number, a series of lines, dots, or numbers which increase as the force increases, as a graph, or in any other fashion which would provide feedback to the individual on how much force they were exerting on the lever or tube. After a resting period, the individual is instructed to exert a specified amount of force on the tube or lever and to maintain that exertion for as long as possible. The testing period ends when the level of force drops below the specified amount. When the visual feedback exhibitor is used, the individual will be able to watch the level of force applied to the lever or arm in order to ensure that it stays above the desired minimum for as long as possible.

In a particularly preferred form of this pain tolerance testing device, the apparatus is adapted for connection to another object described herein via cooperative screws, bolts, or the like. In such an embodiment, it is preferred for the device to include a base connected to the housing by a hinged portion which permits the housing and arm or lever to pivot away from the object to which it is attached and lay flat along the ground. In order to secure the device in the upright position, the housing is pivoted upward and secured into position atop the base using magnets, velcro, screws, bolts, or the like. This base is then the portion of the device that is attached to another object.

In yet another embodiment of the invention, a stabilizing accessory for semi-ambulatory individuals is provided. The accessory assists individuals that desire or need assistance in standing during the performance of testing using the above-described inventions. The accessory generally comprises a frame that provides support to an individual leaning thereon. Preferably, it includes a plurality of legs on one end that extend to the floor and a handle portion at the opposite end. Preferably, the frame provides an individual with enough room to perform the movements required during the testing with a minimum amount of interference. Still more preferably, the accessory is provided with a base that increases the stability of the accessory during testing. In one such embodiment, the accessory comprises two inverted U-shaped members interconnected with at least one crossbar. The crossbar is arched so as to provide an increased movement area to an individual using the accessory. Each of the U-shaped pieces also includes at least one arched crossbar connecting the two leg sections of the U-shaped members. Again, the crossbar is arched outwardly away from an individual using the accessory. The central section of each member has a grip portion adapted to be used as a handle by an individual. The end of each leg section opposite the central section terminates in a base. One particularly preferred base comprises an ANVER (Hudson, Mass.) vacuum or suction cup member equipped with a pivoting suction initiator and release lever. In a particularly preferred embodiment, the accessory is a conventional walker that has been modified to provide an increased area for movement and which includes a suction cup foot at the end of each leg. As with conventional walkers, the device is height-adjustable through a series of push-in detent buttons or projections and cooperative holes that permit the handle portion to telescope into the leg sections. The arched configuration of the crossbars increases an individual's movement area between the two U-shaped members so that the testing can be performed with a minimum amount of interference resulting from use of the accessory. In preferred forms, the stabilizing accessory will be able to be broken down and pivoted together in order to facilitate storage thereof. In this form, each of the individual components may be disconnected and stored or portions of the accessory will be disconnected and the remainder of the device will be pivoted together. For example, the crossbar connecting the two U-shaped members may include a pivoting portion which permits the U-shaped members to be moved into close proximity with one another, thereby decreasing the amount of space required for storage of the accessory.

In use, the accessory is positioned atop or on the floor adjacent one of the invention embodiments. The suction cup levers are then pivoted in order to increase the suction and thereby provide a more secure attachment between the accessory and the floor or device. An individual undergoing testing can then position themself between the two U-shaped members and grasp the handle portion to stand therein. Testing can then proceed as described above for the invention embodiments.

### BRIEF DESCRIPTION OF THE DRAWINGS

FIG. 1 is a perspective view of a preferred oculomotor testing device;

FIG. 2 is a side elevational fragmentary view of a preferred oculomotor testing device illustrating preferred panels of the device;

FIG. 3 is a schematic top view of the switches and electrical wiring of a preferred oculomotor testing device;

FIG. 4 is a cross-section view illustrating a pair of interconnected panels;

FIG. 5 is a perspective view of a two-pad board embodiment of the present invention;

FIG. 6 is a perspective view of a preferred embodiment of the invention designed for non-ambulatory individuals;

FIG. 7 is a perspective view of the embodiment of FIG. 6 in use;

FIG. 8 is a perspective view of a preferred embodiment of the pain tolerance testing apparatus of the present invention;

FIG. 9 is a perspective view of a preferred embodiment of the pain tolerance testing apparatus of the present invention;

FIG. 10 is a side elevational fragmentary view of the interior of the pain tolerance testing unit of the present invention;

FIG. 11 is a perspective view of the embodiment of FIG. 1, together with an accessory useful for stabilizing semi-ambulatory individuals;

FIG. 12 is a perspective view of the stabilizing accessory;

FIG. 13 is a perspective view of the stabilizing accessory in a stored position; and

FIG. 14 is a top plan view of the stabilizing accessory positioned on the embodiment of FIG. 1.

### DETAILED DESCRIPTION OF THE PREFERRED EMBODIMENT

The following description sets forth preferred embodiments of the present invention. It is to be understood, however, that this description is provided by way of illustration and nothing therein should be taken as a limitation upon the overall scope of the invention.

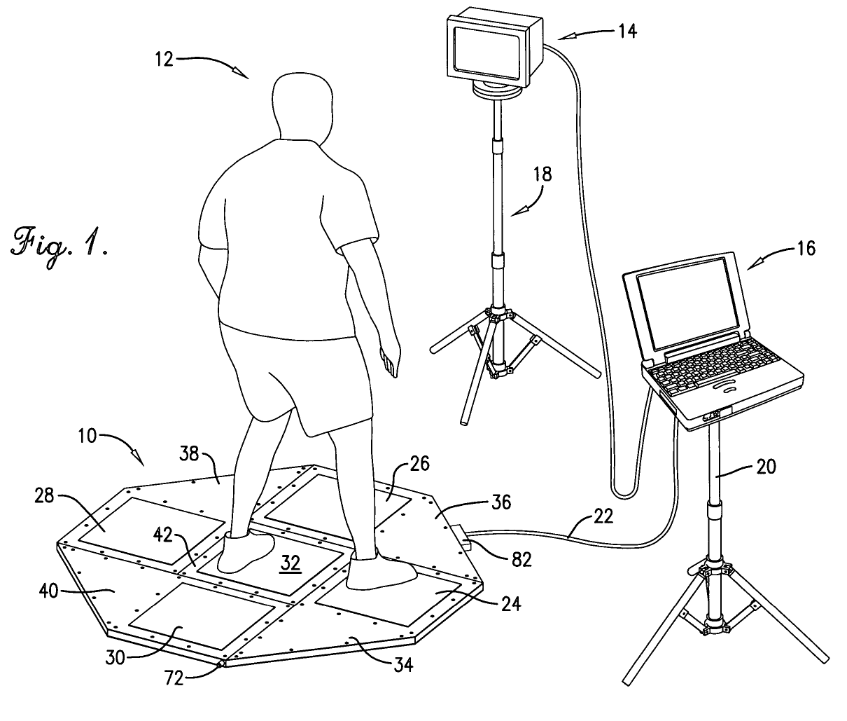

Turning now to the drawings, a preferred oculomotor testing device 10 is illustrated in FIG. 1. A test subject 12 is standing upon the device 10 facing a monitor 14 which is electrically connected to a computing device 16 in the form of a portable computer. Monitor 14 and computing device 16 are positioned atop respective stands 18, 20. Oculomotor testing device 10 is in the shape of an octagon and is also electrically connected to computing device 16 through cord 22. Testing device 10 presents four perimeter pads 24, 26, 28, 30, surrounding a central pad 32. Pads 24, 26, 28, 30, 32 can be of any shape and size and one preferred shape is square.

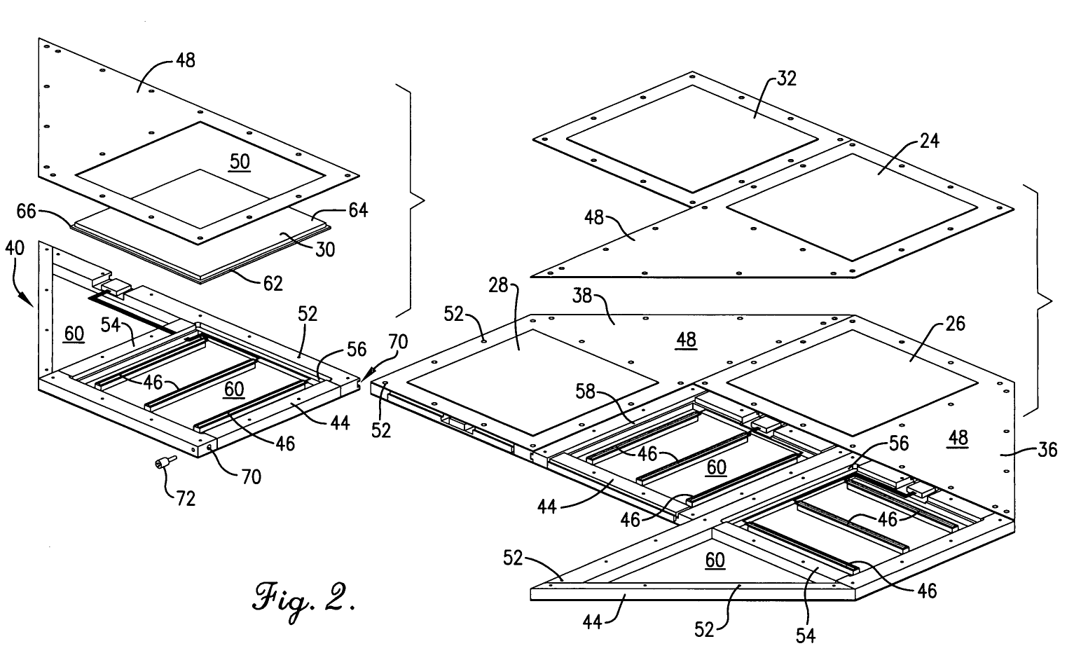

As shown by FIG. 2, each respective perimeter pad sits within a trapezoidal board panel 34, 36, 38, 40 while central pad 32 sits within board panel 42 with the board panels 34, 36, 38, 40, 42 cooperatively forming octagon-shaped testing device 10. Each board panel comprises a perimeter frame 44 which encloses a plurality of switches 46. Preferably, switches 46 are ribbon-style strip switches arranged in parallel and are evenly aligned underneath each of the pads 24, 26, 28, 30, 32. Each board panel further comprises a top sheet 48 having a cut-out portion 50 adapted to surround one of pads 24, 26, 28, 30, 32. Top sheet 48 is secured to frame 44 via a plurality of connecting devices such as screws which extend through top sheet 48 and into frame holes 52.

In the embodiment of FIG. 2 pads 24, 26, 28, 30, 32 are square in shape and there are three switches 46 underneath each pad. These switches 46 run parallel to one another and are substantially evenly positioned underneath each respective pad with two of the switches positioned under opposite ends of a pad with the third switch being centrally positioned under the pad. Frame 44 also includes interior frame member 54 for board panels 34, 36, 38, 40. Switches 46 are bordered on three sides by frame 44 and on the fourth side by frame member 54, thereby creating a square within which switches 46 are housed. The square created by frame 44 and frame member 54 presents a shoulder 56 upon which any one of pads 24, 26, 28, 30 contacts when device 10 is in use. In the case of the perimeter frame 44 for board panel 42, there is no need for an interior frame member as frame 44 is in the shape of a square. However, each side of frame 44 for board panel 42 also includes frame shoulder 58 upon which pad 32 contacts when device 10 is in use.

Board panels 34, 36, 38, 40, 42 may also comprise a bottom sheet 60 which is designed to lie adjacent the ground when device 10 is being used. Thus, board panels 34, 36, 38, 40, 42 have a sandwich-style construction with top sheet 48, together with any respective pad 24, 26, 28, 30, 32, and bottom sheet 60 covering perimeter frame 44, interior frame member 54, and switches 46.

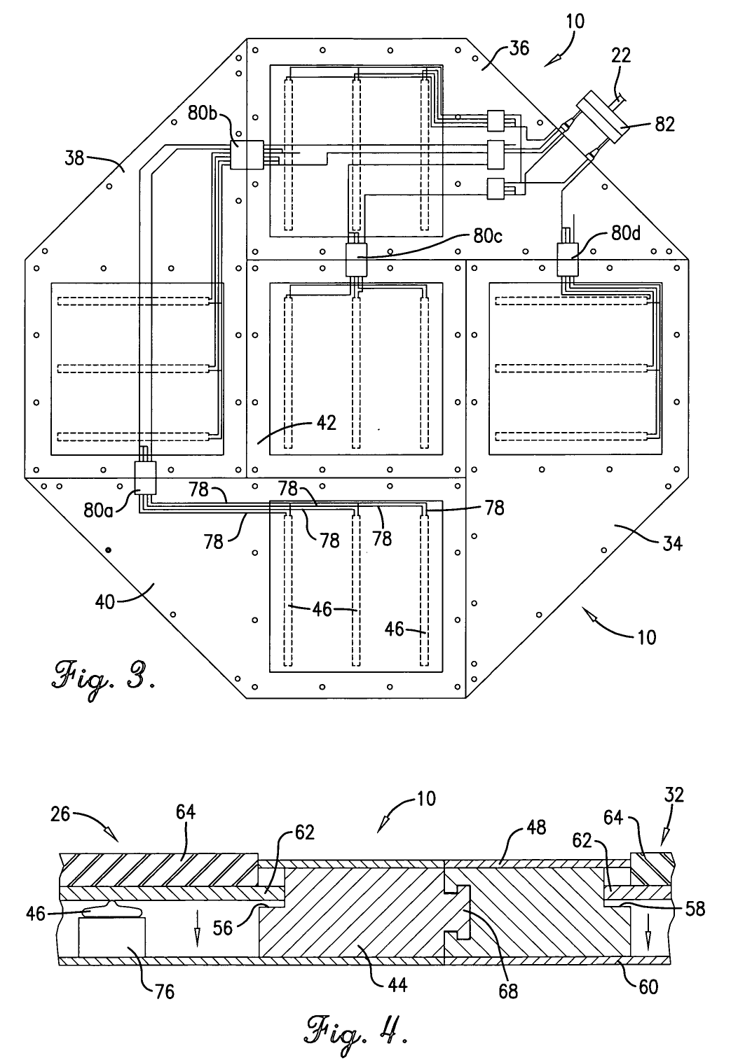

Pad 24, 26, 28, 30, 32 is preferably in the shape of a square and includes a base portion 62 and pad raised portion 64. In preferred forms and as illustrated in FIG. 4, raised portion 64 has a smaller diameter than base portion 62 such that raised portion 64 extends through cut-out portion 50 while the outer edges 66 of base portion 62 are positioned under top sheet 48. FIG. 4 further illustrates dovetail projections 68 which fit into dovetail recesses 70 of adjacent board panels 34, 36, 38, 40, 42 such that the panels cooperatively fit together. Preferably each portion of frame 44 which abuts another portion of frame 44 on an adjacent panel 34, 36, 38, 40, 42 has either a dovetail projection 68 or recess 70 wherein the projection 68 slides into recess 70 to produce a snug fit between adjacent board panels. However, frame 44 of board panel 40 does not have a projection or a recess to couple it with board panel 34 as the adjacent portions of frame on these board panels 40, 34 are merely positioned flush against one another. In preferred forms, board panel 34 includes a projection (not shown) which can be inserted into orifice 72 and secured by locking screw 74 which serves to secure board panels 34, 40 together. Also illustrated in FIG. 4 is a block 76 upon which switches 46 sit. Pad base portion 62 rests on top of switch 46, which, in its inactive state is open or off. However, when pad 26 is depressed during testing by having a test subject's weight placed thereon, base portion 62 compresses switch 46 into an active state which is closed or on, thereby completing the circuit and sending a signal to computing device 16. When a test subject's weight is placed on a pad 24, 26, 28, 30, 32, base portion 62 is depressed until it contacts shoulder 56 which is placed at a level which permits switch 46 to be activated.

FIG. 3 illustrates a schematic of the wiring of device 10. As shown, each switch 46 is connected in parallel with the other switches within each respective board panel 34, 36, 38, 40,42 and the electrical leads 78 from these switches converge at connectors 80a, 80b, 80c, 80d, located between adjacent board panels and eventually all meet at interface 82 which is connected to cord 22.

In one preferred method of operation, test subject 12 stands on central pad 32 facing monitor 14. Computing device 16 then sends a signal to monitor 14 to provide a visual stimulus for test subject to see and react to. Each stimulus will be associated with a specific instruction for the test subject 12 to follow. For example, the visual stimulus maybe a color which prompts the test subject 12 to step off of pad 32 and onto a pad having the same color as the stimulus and then return to pad 32. Such a process would be termed a single movement with a test period being made up of a plurality of movements. The results of such a test process could be provided for any particular parameter including reaction time, movement time, and combinations thereof. For example, for any test period, the total time taken by the test subject to complete the test period could be provided as could the total of the reaction time or the movement time. Such results could further be divided up into averages and means and compared to other populations of test subjects or standards for any population subset as well as with previous testing periods for the same or even a different individual. Moreover, the results could include the type and incidence of any errors committed for any specific locomotor response to a specific stimulus. For example, if a test subject was prompted to move to pad 34 but consistently moved to pad 36 in response to the prompt yet always moved correctly to pad 38 in response to the prompt to do so, data regarding this difference could be collected and reported. If desired, the time it took the test subject to perform the incorrect movements could be factored out of the results entirely so that such time would not contribute to the testing results.

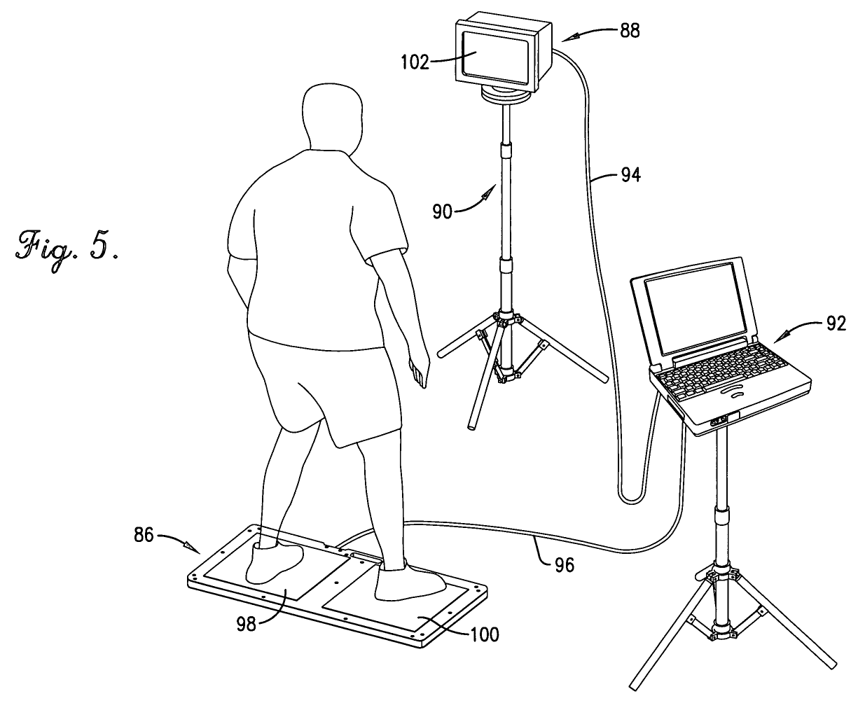

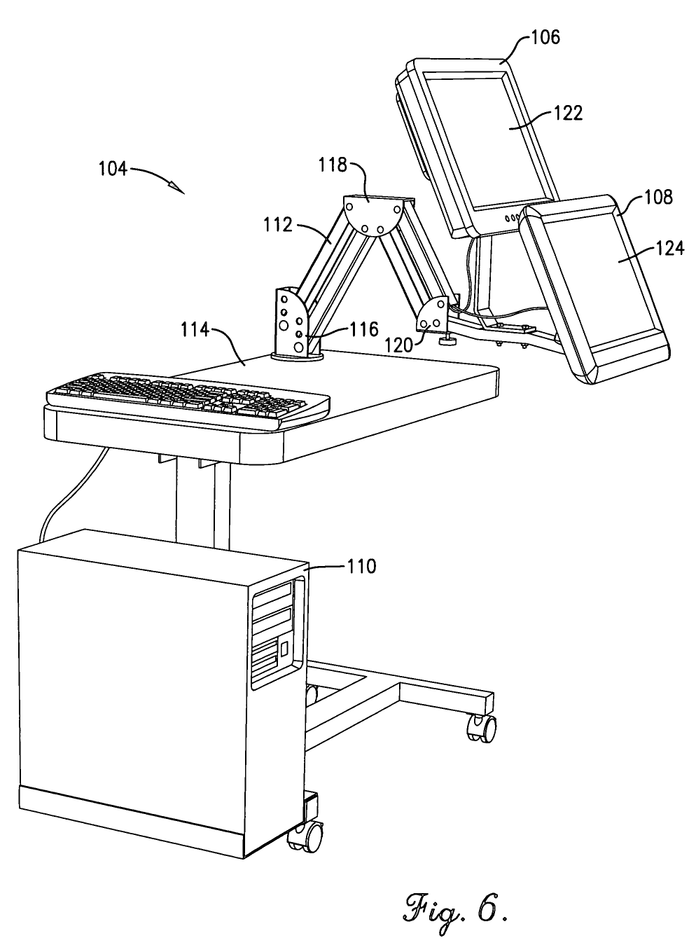

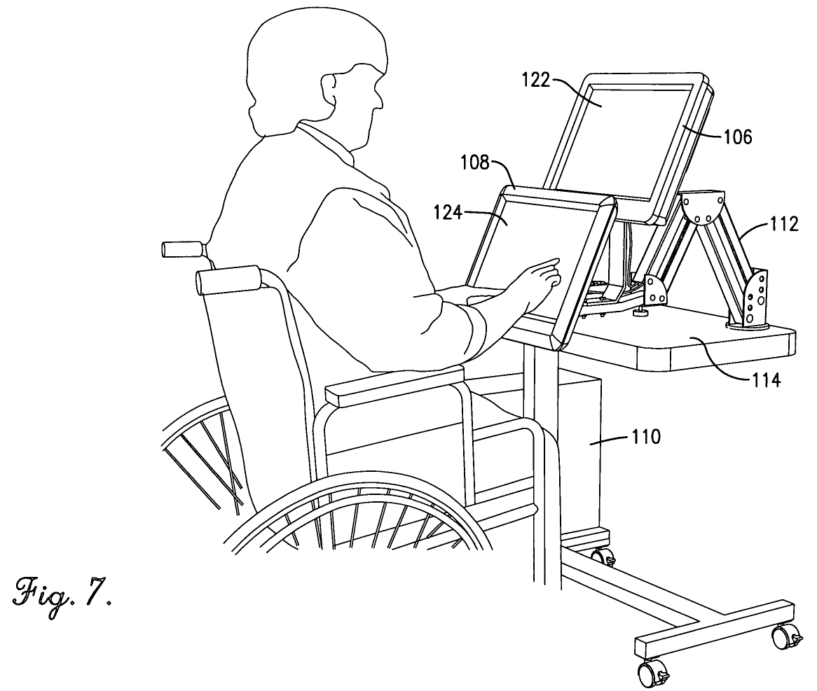

FIG. 5 illustrates a two-pad embodiment 86 of the present invention that is similar in construction to the oculomotor testing device 10. In preferred forms, this embodiment also comprises a visual stimulus exhibitor 88, preferably a video monitor, mounted on an adjustable-height stand 90 and a computing device 92 adapted for intercommunication with the exhibitor 88 and the embodiment 86. One preferred method of facilitating intercommunication is by electrically connecting the computing device 92 with exhibitor 88 and embodiment 86 via wires 94, 96. Embodiment 86 is preferably used to measure reaction time in response to a visual stimulus.

In one preferred method of using embodiment 86, embodiment 86, exhibitor 88, and computing device 92 are provided. Computing device 92 is adapted for intercommunication with embodiment 86 and exhibitor 88. A test subject stands with their left foot on pad 98, which is preferably red and with their right foot on pad 100, which is preferably black. Computing device 92 sends a signal to the exhibitor 88 to begin a countdown prior to the onset of the testing period. Once the countdown reaches zero, screen 102 on exhibitor 88 turns white. Computing device 92 then sends a signal to exhibitor 88 at a random time after screen 102 has turned white. In response to the signal, the exhibitor will display a stimulus which instructs the individual to lift a foot off of one of the pads 98, 100. The stimulus is preferably a color corresponding to the color of one of the two pads 98, 100. In this manner, if screen 102 displayed the color red and pad 98 was red, the individual would lift their left foot off of pad 98 as fast as possible after the color was displayed. The elapsed time between the onset of the stimulus and the locomotor response of lifting the foot would be recorded by the computing device 92 and would be representative of the reaction time. This method could be repeated for any number of repetitions in a testing phase and averaged in order to provide a representative sample of the individual's reaction time. Preferably, the testing phase would have at least two repetitions (one for the lifting of each foot) which would induce the individual to lift each foot at least one time and these repetitions would be random in order. For example, in order to get one repetition for each side, the testing phase would need to have two separate and different displays, with each different display corresponding to one of the pads and consequently, the lifting of each foot one time. However, such a testing phase may include more than one repetition for each side in order to prevent the tested individual from anticipating what the next stimulus displayed will be and moving prior to the onset of the stimulus or quicker in response to the stimulus. Thus, for a testing phase to consist of one repetition to each side, there may be a total of three or more repetitions (e.g. two to one side followed by one to the other). Preferably, the number of repetitions for each foot (or pad) is equal. Still more preferably, each display related to a specific oculomotor response is repeated at least four times so that each foot is lifted from each pad 98, 100 at least four times to make up a testing phase. Even more preferably, between about four and twenty responses make up a testing phase. In some preferred methods, if the individual lifted the wrong foot from the pads 98, 100, the individual would have to repeat that repetition at some time during the testing phase. It is also preferred to have the displays generated randomly in order to prevent the individual being tested from anticipating the next correct response.

FIG. 6 illustrates an embodiment of the present invention that is especially useful for non-ambulatory individuals. Apparatus 104 preferably includes visual stimulus exhibitor 106, preferably a conventional computer monitor, touch screen 108, and computing device 110, preferably a computer. Computing device 10 is adapted for intercommunication between the exhibitor 106 and touch screen 108. Touch screen 108 is a conventional touch screen such as those sold by Elo TouchSystems (Fremont, Calif.) wherein touching a location on a screen transmits data to a computing device. Touchscreen 108 and exhibitor 106 are mounted on arm 112 attached to table 114. Preferably, arm 112 is adjustable through a series of hinges 116, 118, 120 so as to accommodate individuals of different heights. It is also preferred that exhibitor 106 and touchscreen 108 are located in different sight planes so that an individual using the apparatus must either memorize the touch location(s) on the touchscreen 108 or divert their sight from the exhibitor 106 to the touchscreen 108 in order to respond to the stimulus displayed on screen 122.

In use, apparatus 104 is useful in the same applications and can measure the same parameters as embodiment 86. In a preferred method of using apparatus 104, an individual sits facing exhibitor 106 and touch screen 108. Computing device 110 sends a signal to exhibitor 106 which begins a countdown to the onset of the first visual stimulus. The countdown may be either audible or visual. Once the count reaches zero, the display on screen 122 should be white prior to displaying a visual stimulus on screen 122. The computing device 110 then sends a signal to exhibitor 106 to display a visual stimulus on screen 122. This signal is sent at a random time so as to reduce any errors resulting from the individual anticipating the timing of the stimulus display. Moreover, the signal sent by computing device 110 is random as to which selected stimulus will be displayed on screen 122, provided that the stimulus corresponds to one area or portion on the face 124 of touchscreen 108. The visual stimulus will be the same as described above for other embodiments of this invention. The individual using apparatus 104 will view the stimulus and respond by touching the area or portion of the face 124 of touchscreen 108 corresponding to the stimulus as quickly as possible. For example, if face 124 of touchscreen 108 had one area thereon which was red and one area thereon which was black, and the visual stimulus was red, the individual using the apparatus would move one of their hands as quickly as possible to touch the red area. Once touchscreen 108 is touched, it sends a signal to computing device so that computing device can determine if the correct area was touched first and measure and/or record the desired reaction parameter. As with the other embodiments of this invention, a testing phase can consist of as many repetitions as desired with preferred numbers of repetitions being described above in relation to other embodiments.

In an alternative use of apparatus 104, the individual using the apparatus may need to use both hands to touch the face 124 of screen 108, with the left hand being responsible for touching the left portion of face 124 and their right hand being responsible for touching the right portion of face 124. In this manner, if the left side of face 124 of touchscreen 108 had one area thereon which was red and the right side of face 124 had one area thereon which was black, and the visual stimulus was red, the individual using the apparatus would move their left hand as quickly as possible to touch the red area. Of course, the opposite hand would be used if the stimulus displayed were black.

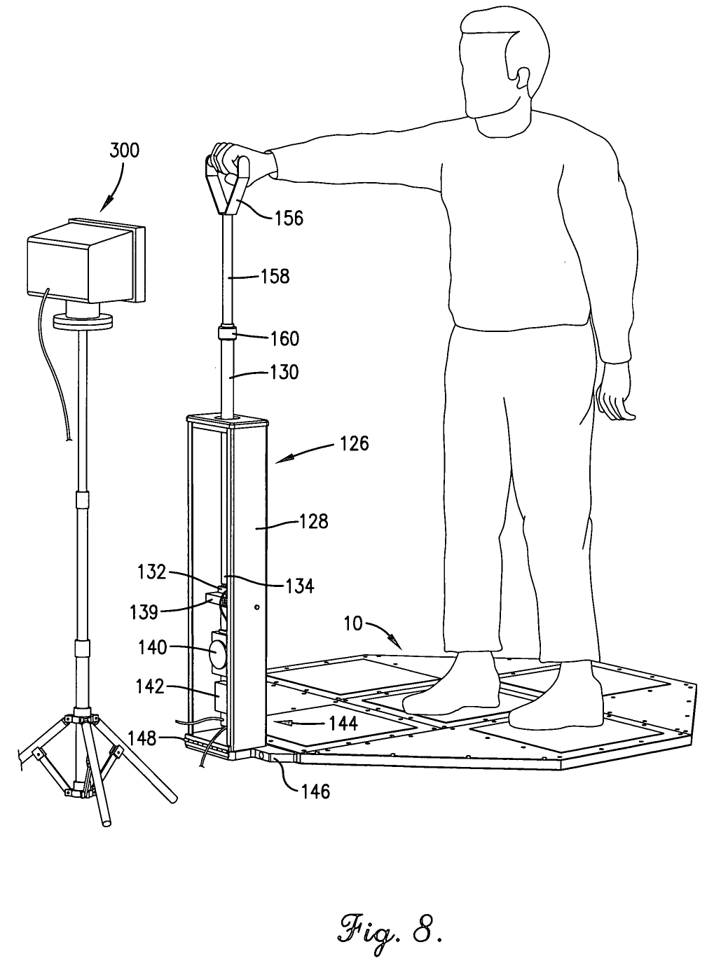

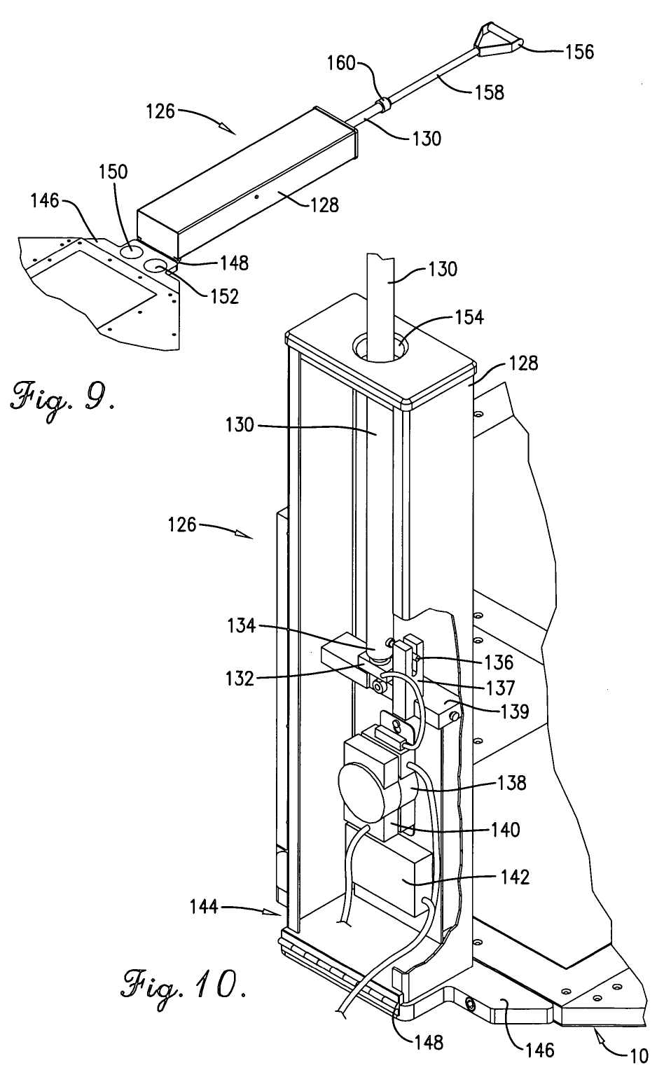

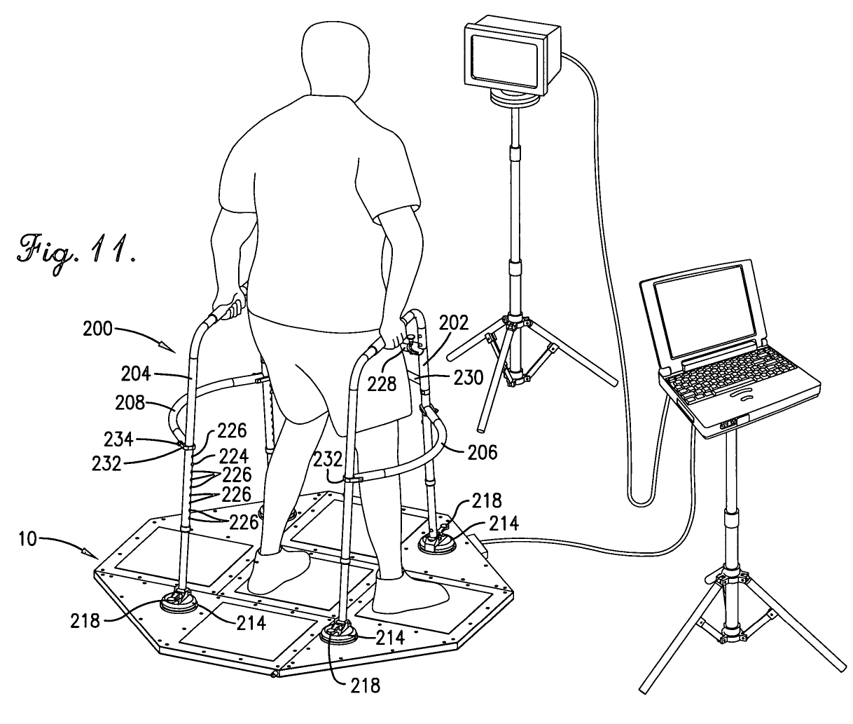

The pain tolerance testing device 126 of the invention is depicted in FIGS. 8-10 and generally comprises housing 128 and tube 130. Housing 128 substantially encloses lower portion of tube 130 which is connected to load cell 132 at its lower end 134 which also includes an anti-rotate pin 136 connected to anchor 137 which prevents tube 130 from rotating within housing 128. Anchor 137 is further attached to support bar 139 which is secured in housing 128. Load cell 132 is adapted for intercommunication with a first ADAM module 138 which is adapted to intercommunicate with a second ADAM module 140. As shown in FIG. 10, first ADAM module 138 and second ADAM module 140 may be arranged in a stacked fashion although any arrangement is possible. ADAM module 140 is preferably adapted to intercommunicate with a computing device (not shown). Power to apparatus is supplied through power supply 142 which may also be connected to a source of electricity (not shown). In preferred forms, lower end 144 of housing 128 is metal and includes base portion 146 is adapted for attachment to another object such as device 10 using screws or the like. It is also preferred for lower end 144 to connect with base portion 146 via hinge 148 which permits device 126 to pivot and lie flat on the ground as depicted in FIG. 9. Base 146 may also include magnets 150, 152 which help to secure device 126 in an upright position when in use. Of course, other forms of securing device 126 in an upright position are contemplated and include straps, screws, bolts, latches, hook and loop type fasteners, snaps, and the like. Tube 130 extends through an opening 154 in housing 128 and terminates in handle 156. As shown in FIGS. 8 and 9, tube 130 has an upper, extendable portion 158 adapted to telescope within tube 130. Once a desired height for portion 158 is determined, hand collet 160 is tightened to prevent further telescoping of portion 158.

In use, device 126 is set up in the upright position such as is illustrated in FIG. 8 and attached to another object such as device 10 to prevent it from being vertically displaced during use. Device 126 is further adapted to intercommunicate with a computing device. The user grasps handle 156 with their palm side down and telescopes tube portion 158 from tube 130 to the desired height, preferably so that the user's shoulder is at a 90° angle relative to their body. Hand collet 160 is then tightened and the user will observe a visual feedback display 300 such as a conventional computer monitor that is adapted for intercommunication with a computing device. When prompted, the user will exert the maximum possible force in pulling upward on handle 156 and this value will be transmitted to the computing device and used to compute the individual's maximum strength. It is noted that this force is measured by load cell 132 without any relative movement of tube 130. In other words, tube 130 remains stationary despite the force exerted by the user. The feedback display will exhibit a real-time depiction of the force exerted on load cell 132 which can be observed by the user. Once the maximum strength is determined and after a brief rest period, the user is instructed to again pull up on handle 156 and to maintain a specified amount of exerted force on the handle 156 for as long as they can. Once the amount of force drops below the specified amount, the repetition is complete. It is preferable that the specified amount of force is a percentage of the user's maximum strength. In order to provide the user with feedback regarding how much force is being exerted on handle 156, the feedback display will provide an indication of this amount of force by way of a gauge, number, graph, or other visually distinct depiction that will show the user, in real time, where the threshold that must be met is relative to the amount of force being exerted. For example, the individual may be informed that the display will show, in numerical form, the percentage of the force being exerted relative to the individual's maximum strength. The individual will then be informed that if the force exerted drops below a certain number, the repetition will end and the time elapsed between the beginning of exertion and the dropping below the threshold value recorded and used to ascertain that individual's tolerance for pain, among other things.

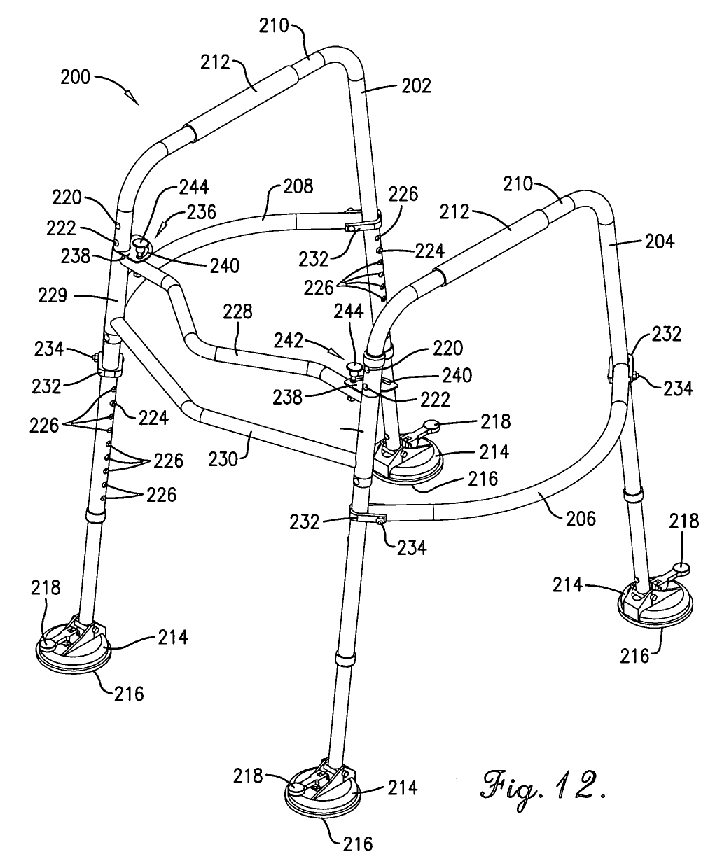

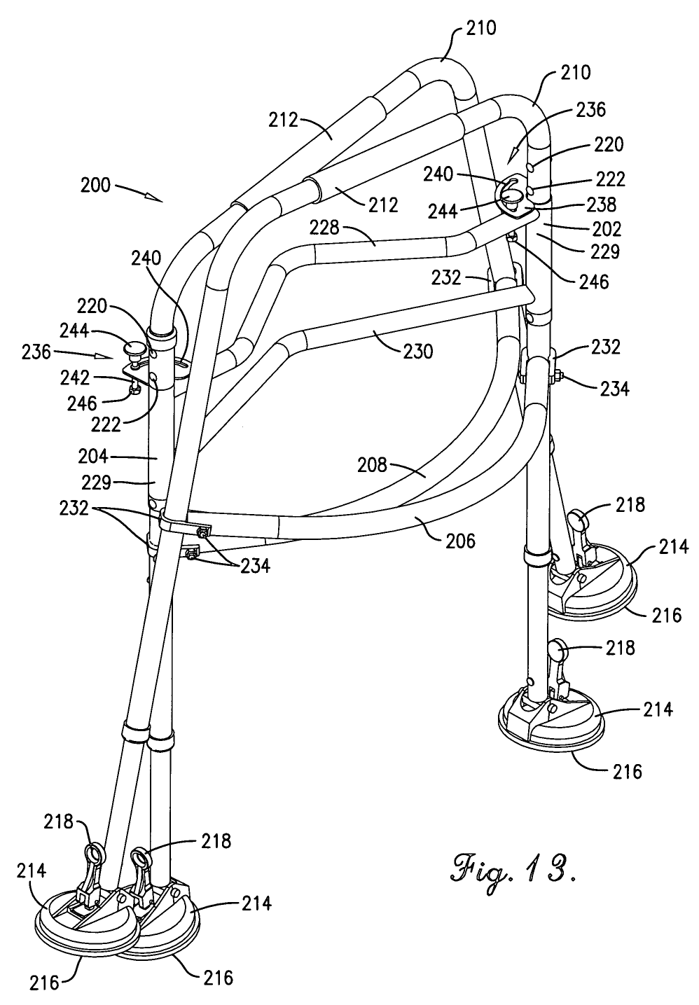

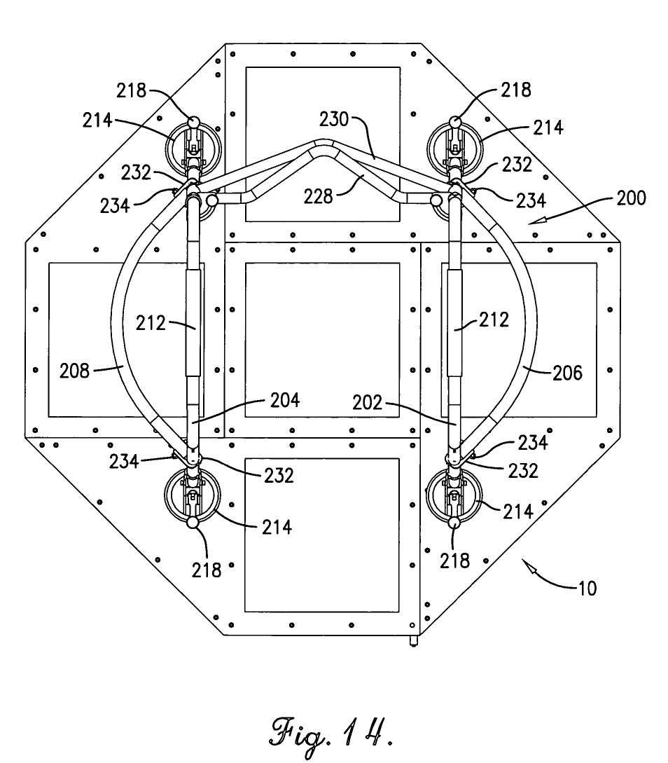

An accessory 200 useful with the embodiments described above is depicted in FIGS. 11-14. In FIG. 11, accessory 200 is shown positioned atop oculomotor testing device 10, it being understood that accessory 200 may also be atop or on the floor adjacent two-pad embodiment 86 or pain tolerance testing device 126. Accessory 200 includes two inverted U-shaped members 202, 204, each including an arched crossbar piece 206, 208 interconnecting the legs of each member 202, 204 near the midpoint thereof. The legs of U-shaped members 202, 204 interconnect through section 210 at one end having a grip portion 212 thereon. The end of the legs opposite section 210 terminate in feet 214, preferably having a flexible rubber sole 216 on the bottom thereof. Feet 210 are preferably suction cup-like and have a device 218 that pivots in one direction to increase the vacuum pressure (such as is shown in FIG. 11) created by the suction cup and in the opposite direction (such as is shown in FIG. 13) to decrease the vacuum pressure. Section 210 is height adjustable through a series of cooperative push in buttons 220 that are adapted to project through holes 222 positioned at different heights. Buttons 220 on section 210 are pushed in and section 210 telescopes into the legs of members 202, 204 until the buttons 220 snap outwardly and extend through one of the holes 222. Each of the legs of the U-shaped members 202, 204 may also be height-adjustable through a similar series of projections 224 and holes 226 that permit telescoping of a lower section of each leg. Members 202, 204 may further be connected to one another through crossbars 228, 230, each of which are arched outwardly and extend between one leg of each member 202, 204. In preferred forms, crossbars 228, 230 include a sleeve 229 in which members 202, 204 can rotate. Crossbars 206, 208, 228, 230 can be connected to members 202, 204 in any conventional manner including welding, bolts, glue, tension, push-in snap out detents, screws, or conventional bracket and fastener assemblies. One preferred embodiment presents crossbars 206, 208 that are attached to members 202, 204 via a bracket 232 and bolt 234 assembly while crossbars 228, 230 are welded to members 202, 204 at their attachment points. In order to reduce the amount of space required for storage of device 200, a locking hinge 236 is provided at each end of crossbar 228. Hinge 236 can be any conventional locking hinge but preferably includes bracket 238 with slide track 240. Spring-loaded bolt 242 having push cap 244 at one end is inserted into slide track 240 whereupon nut 246 is threaded onto bolt 242. Spring-loaded bolt 242 has a small diameter upper section 248 proximate to pushcap 244 and a large diameter lower section 250 proximate to nut 246. When section 250 is in registration with track 240, no relative movement between bolt 242 and track 240 can occur. Applying pressure to push cap 244 forces spring-loaded bolt 242 downward so that the large diameter section 250 is moved down and away from slide track 240 and small diameter section 248 is moved into registration with slide track 240. When section 248 is in registration with track 240, the attached U-shaped member can pivot or fold into itself by rotating within sleeve 229 such that member 202 or 204 is substantially parallel rather than substantially perpendicular to crossbar 228. As shown by FIG. 13, such an assembly greatly reduces the space necessary to store accessory 200.

1. An oculomotor testing device comprising:
a plurality of board panels, each board panel comprising a plurality of switches electrically connected to a computing device, each of said switches being in a first position in an inactivated state;
a pad associated with each of said board panels, said pad overlying said switches whereby when weight is applied to said pad, at least one of said switches shifts to a second position.

2. The device of claim 1, said first position being selected from the group consisting of open and closed.

3. The device of claim 2, said second position being open when said first position is closed and closed when said first position is open.

4. The device of claim 1, said second position sending input to said computing device.

5. The device of claim 1, further including a stimulus exhibitor.

6. The device of claim 5, said stimulus generator being a monitor.

7. The device of claim 5, said stimulus generator displaying colors.

8. The device of claim 1, said plurality of board panels including connectors.

9. The device of claim 8, said connectors comprising cooperative dovetail projections and recesses.

10. The device of claim 1, said board panels further comprising a perimeter frame.

11. The device of claim 10, said frame enclosing said switches.

12. The device of claim 10, said frame including an egress for said electrical connection to said computing device.

13. The device of claim 10, said frame having a top cover and a bottom cover.

14. The device of claim 13, said top cover and said bottom cover comprising aluminum.

15. The device of claim 13, said top cover presenting a cut-out portion, at least a portion of said pad adapted to fit in said cut-out portion.

16. The device of claim 10, said frame including a shoulder, said pad being spaced from said shoulder when said switch is in said first position.

17. The device of claim 16, said pad being adjacent said shoulder when said switch is in said second position.

18. The device of claim 1, said pad including a non-skid surface.

19. The device of claim 1, said plurality of switches being connected in parallel.

20. The device of claim 1, at least one said switch returning to said first position when the weight is removed from said pad.

21. The device of claim 20, said switch sending data to said computing device when said switch returns to said first position.

22. The device of claim 1, said computing device adapted to measure a response parameter selected from the group consisting of reaction time, movement time, and combinations thereof.

23. The device of claim 1 further including a detachable upright frame, said frame including a base and a handle portion.

24. The device of claim 23, said frame comprising a pair of inverted U-shaped members interconnected with a crossbar.

25. A method of measuring a response parameter of an individual, said parameter being selected from the group consisting of reaction time, movement time, and combinations thereof, said method comprising the steps of:
a) providing a visual stimulus;
b) causing a locomotor response to said stimulus, said locomotor response generating data, said data representative of at least one of said parameters; and
c) collecting said data in order to measure said parameter.

26. The method of claim 25, further including the step of repeating steps a-c a first plurality of times.

27. The method of claim 26, said plurality being at least 10.

28. The method of claim 27, said plurality being at least 20.

29. The method of claim 28, said method being between about 25-50.

30. The method of claim 26, further including the step of processing said data to give a total elapsed time of at least one of said parameters.

31. The method of claim 25, said locomotor response consisting of the individual moving from a first position to a second position.

32. The method of claim 31, said locomotor response further consisting of the individual moving from said second position back to said first position.

33. The method of claim 25, further including the step of comparing said collected data with another set of collected data, said other set of collected data being selected from the group consisting of a known standard, a specific population of individuals, a previously collected set of data from the individual, and a previously collected set of data from another individual.

34. The method of claim 25, further comprising the step of using said data for a type of testing selected from the group consisting of athletic testing, geriatric evaluation, physical therapy progression, locomotor rehabilitation, drug impairment evaluation, vestibular testing for inner ear disorders, multiple sclerosis progression, and child locomotor skills.

35. The method of claim 26, including the step of repeating steps a-c a second plurality of times after said first plurality of times.

36. The method of claim 35, including the step of having a first rest period between said first and second plurality of times.

37. The method of claim 36, said first rest period lasting at least 1 minute.

38. The method of claim 37, said first rest period lasting between 1-5 minutes.

39. The method of claim 35, including the step of repeating steps a-c a third plurality of times after said second plurality of times.

40. The method of claim 39, including the step of having a second rest period between said second and said third plurality of times.

41. The method of claim 40, said second rest period lasting at least 1 minute.

42. The method of claim 41, said second rest period lasting between 1-5 minutes.

43. The method of claim 25, wherein the visual stimulus presents the individual with a choice between at least two locomotor responses with a single locomotor response being correct and the remaining locomotor responses being incorrect.

44. The method of claim 43, step c further including the step of collecting data regarding correct and incorrect responses.

45. The method of claim 44, said data including the frequency of a specific incorrect response.

46. An oculomotor testing apparatus comprising:
a stimulus exhibitor;
a touch screen; and
a computing device adapted for communication with said stimulus exhibitor and said touch screen.

47. An apparatus comprising:
a computing device;
a visual feedback exhibitor in communication with said computing device; and
a force receiving assembly in communication with said computing device, said force receiving assembly including a force-receiving member operatively connected to a force measuring assembly, wherein force exerted on said force receiving member is measured by said force measuring assembly, transmitted to said computing device, and visually represented on said visual feedback exhibitor.

48. A stabilizer apparatus comprising:
a pair of U-shaped frame members, each member comprising a pair of legs interconnected at one end by a handle portion, said other end of each of said legs terminating in a base, each said member also including a reinforcing bar spanning between said pari of legs on each said member, said reinforcing bar presenting an arched shape; and
a crossbar interconnecting one leg of each said member, said cross bar presenting an arched shape.
